(19) United States
(12) Patent Application Publication
(10) Pub. No.: US 2003/0143206 A1
(43) Pub. Date: Jul. 31, 2003

(54) Method of treatment of anorectal disorders

(76) Inventor: Denis King, Kogarah (AU)

Correspondence Address: Nixon & Vanderhye, 8th Floor, 1100 North Glebe Road, Arlington, VA, 22201-4714, US

(21) Appl. No.: 10/204,548

(22) PCT Filed: Feb. 21, 2001
(86) PCT No.: PCT/AU01/00172

(30) Foreign Application Priority Data
Feb. 22, 2000 (AU) PQ 5777

(51) Int. Cl. A61K 48/00; A61K 38/44

(52) U.S. Cl. 424/93.2; 424/94.4; 514/44

(57) ABSTRACT

A method of treatment of an anal disease, said method including the step of increasing the level of at least one isoform of the enzyme nitric oxide synthase in the cells of the ano-rectal region of a subject in need of such treatment.

### FIELD OF THE INVENTION

The present invention relates to a method of treating ano-rectal conditions, such as anorectal fissure, anal ulcers, haemorrhoidal disease and levator spasm.

### BACKGROUND ART

Ano-rectal conditions such as anal fissure/ulcer, haemorrhoids and levator spasm have been shown or at least postulated to be based on increased pressure in the lower part of the internal anal sphincter.

While none of these conditions are pre-malignant, they can be distressing and difficult to treat.

An anal fissure Or anal ulceration is a tear of the lining of the anal canal at or near the anal verge. Haemorrhoids arise due to enlargement of the anal cushions. The cushions normally function to assist in closure of the anal canal. In certain individuals, however, the anal cushions become enlarged with an increased venous component which can develop thromboses.

The internal sphincter is an involuntary muscle continuous with the inner circular layer of rectal muscle. In anal fissure/ulcer and haemorrhoids, a high intra anal pressure is often found as a result of internal sphincter hypertonicity. Intra-anal pressure measurements obtained from people suffering from anal fissure/ulcer disease or haemorrhoidal disease show an exaggerated pressure response to a variety of stimuli. Indeed, the resting pressure may even exceed arterial pressure. The abnormally high intra-anal pressure is responsible for the associated pain of the fissure/ulcer or haemorrhoid and is presumed to prevent healing.

A proportion of the conditions described above will settle spontaneously and much of the treatment used is for symptomatic relief, including topical application of local anaesthetic agents and corticosteroids. These products, however, do not treat the underlying cause of the problem, that is, the high resting pressure in the anal canal due to internal sphincter hypertonicity.

There are several methods to deal with the underlying cause, particularly in the treatment of anal fissure. The best results have been reported with surgical division of the lower part of the internal anal sphincter. This results in reduced intra anal pressure and allows healing of anal fissures in around 95% of patients. There is, however, a definite incidence of distressing and sometimes uncomfortable incontinence of flatus or faeces.

Nitric oxide (NO) has been shown to be an inhibitory transmitter to muscle (see Rappan Fla., “Nitric oxide pathway in recto anal inhibitory reflex of opossum internal anal sphincter”. Gastroenterology Vol 103 1992: Pages 43-45; Chakder et al, “Release of nitric oxide by activation of non adrenergic, non cholinergic neurones of internal anal sphincter” American Journal of Physiology Vol 264: G7 to G12 1993; O'Kelley et al, “Nerve mediated relaxation of the internal anal sphincter: The role of nitric oxide” Gut Vol 34: 689-693, 1993). Organic nitrates such as nitroglycerine (GTN) have been shown to release nitric oxide and when applied topically to the anal region have been shown to decrease internal anal sphincter pressure and allow healing of anal fissures (King DW “Topical Glyceryl Trinitrate in the Management of Anal Fissures, Sydney Colorectal Surgical Society Jul. 1993).

The problem with the use of topically applied organic nitrates is that sufficient amounts of nitric oxide are released into the circulation to produce headache in a significant number of patients, in some to the point where they are unable to tolerate the medication.

Another apparent problem is the relatively short period of action of locally applied organic nitrates which means they must be applied at least three times a day and even then produce healing of anal fissures in only 50% of patients.

Another method of producing relaxation of the internal anal sphincter is to inject Botulinum toxin into the space between the internal and external anal sphincters related to that part of the internal anal sphincter below the dentate line. Botulinum causes muscle relaxation for up to six months and has been found to produce healing of anal fissures in 90% of patients.

Botulinun toxin produces a much longer period of relaxation and is associated with significantly better results than GTN but requires an injection which in some people dictates the need for general anaesthesia. It is an expensive substance but at the doses used for the treatment of minor anal conditions there have been no recorded side effects.

The present invention provides an effective method of treatment of ano-rectal conditions which overcomes the problems associated with the prior art.

### SUMMARY OF THE INVENTION

According to a first aspect, the present invention provides a method of treatment of an anal disease, said method including the step of increasing the level of at least one isoform of the enzyme nitric oxide synthase in the cells of the ano-rectal region of a subject in need of such treatment.

By increasing the level of nitric oxide synthase (NOS) activity, cellular production of nitric oxide in said ano-rectal region is induced.

In one embodiment of the invention, the level of NOS in the cells of the ano-rectal region is increased by the introduction of a NOS enhancing factor to said cells.

Preferably, the NOS enhancing factor is a nucleotide sequence encoding at least one isoform of nitric oxide synthase. In this embodiment, the nucleotide sequence encoding the at least one isoform of NOS may be isolated and purified from an appropriate cell or cell line.

The nucleotide sequence may be isolated using any of a variety of procedures. For example, one method includes the step of direct functional expression of NOS cDNA following the construction of a NOS-containing cDNA library in an appropriate expression vector system. Construction of appropriate cDNA libraries can be performed by standard techniques well known in the art see for example Maniatis et al., J. Molecular Cloning: A Laboratory Manual (Cold Spring Harbour Laboratory, Cold Spring Harbour, N.Y., 1982).

By way of example only, cDNA encoding NOS may be isolated and purified from macrophages, macrophage-like cells and macrophage cell lines as disclosed in U.S. Pat. No. 5,766,909, the contents of which are herein incorporated by reference. It will be apparent to those skilled in the art, however, that the cDNA encoding the at least one isoform of NOS may be isolated from other types of libraries. Another example of the isolation of cDNA encoding human tissue inducible NOS is described in U.S. Pat. No. 5,882,908, the contents of which are herein incorporated by reference.

Preferably, the cDNA is isolated from cells or cell lines having NOS activity. It is further preferred that such cells or cell lines are mammalian in origin and more preferably, human in origin.

In another embodiment, the nucleotide sequence encoding NOS may be isolated from a suitable genomic DNA library. Construction of genomic libraries may be performed by standard techniques see. for example Maniatis et al., J. in Molecular Cloning: A Laboratory Manual (Cold Spring Harbour Laboratory, Cold Spring Harbour, N.Y., 1982).

Alternatively, the nucleotide sequence is mRNA encoding the at least one isoform of NOS

In a further embodiment, the isolated nucleotide sequence encoding the at least one isoform of NOS may be recombinantly expressed by molecular cloning into a vector. The vector, which preferably contains a suitable promoter and other appropriate transcription regulatory elements, may then be transferred into a host cell to produce recombinant NOS.

Preferably the vector includes selectable markers, restriction enzyme sites and a potential for high copy number. Further, promoters/enhancers may also be used to control expression of the nucleotide sequence of the at least one isoform of NOS. Promoter activity may be tissue specific or alternatively, inducible by a metabolic product or administered substance.

Suitable expression vectors include, but are not limited to, plasmids, avian retroviral vectors, murine retroviral vectors, adenoviral vectors, herpes viral vectors and non-replicative pox viruses. It is preferred that the vector is replication defective. In this regard, replication defective recombinant viruses may be generated in packaging cell lines that produce only replication defective viruses.

In a preferred embodiment, the vector includes a DNA virus and preferably, a recombinant, replication defective adenovirus. In this embodiment, the adenovirus may be rendered replication-defective by deletion of the E1A and E3 regions of the genome. The E1A is the first gene to be transcribed upon nuclear entry into a host cell and is essential for viral replication. The E3 region appears not to be required for viral growth in culture and deleting it from the genome simply enables a larger DNA insert to be incorporated into the vector.

Any of the methods known in the art for the insertion of polynucleotide sequences into a vector may be used. See for example, Sambrook et al., Molecular Cloning: A Laboratory Manual, Cold Spring Harbour Laboratory, Cold Spring Harbour, N.Y. (1989) and Ausubel et al., Current Protocols in Molecular Biology, J. Wiley & Sons, N.Y. (1992).

The expression vector may be introduced into the cells of the ano-rectal region via any one of a number of techniques including but not limited to transformation and transfection.

In a further embodiment, the nucleotide sequence encoding the at least one isoform of NOS may be introduced into the host cell by lipofection. This embodiment may involve the steps of inserting the nucleotide sequence into a suitable plasmid and introducing the plasmid into a liposome. The liposome acts as a transfection or a transformation system to introduce the nucleotide sequence into the host cells.

Multiple isoforms of the nitric oxide synthase enzyme exist which are generally classified into two broad categories:

1) constitutive

2) inducible

These classes of NOS enzymes vary considerably in their size, amino acid sequence, activity and regulation. By way of example, cells such as brain and vascular endothelial cells express constitutive NOS isoforms whereas macrophages and vascular smooth muscle cells express an inducible NOS. The nucleotide sequences of several isoforms of NOS have been isolated and described in U.S. Pat. Nos. 5,766,909 and 5,882,908

In one embodiment, a nucleotide sequence encoding an inducible isoform of NOS is inserted into a suitable expression vector for subsequent introduction into the cells of the ano-rectal region of a subject. Alternatively, a nucleotide sequence encoding a constitutive isoform of NOS is inserted into a suitable expression vector for subsequent introduction into the cells of the ano-rectal region of a subject.

In a further embodiment, the nucleotide sequence encodes vascular smooth muscle cell inducible NOS.

In another embodiment, the nucleotide sequence encodes brain cell constitutive NOS.

In a further embodiment, the nucleotide sequence encodes vascular endothelial cell constitutive NOS.

Preferably, a nucleotide sequence encoding a single isoform of NOS is introduced into the cells of the ano-rectal region. However, it is envisaged that nucleotide sequences encoding multiple isoforms of NOS may be introduced into the cells of the ano-rectal region.

The expression vector carrying the nucleotide sequence encoding the at least one isoform of nitric oxide synthase (vector construct) may be combined with a pharmaceutically acceptable carrier for topical application to the skin of the perianal region or the lining of the anal canal. Alternatively, the vector may be injected directly into the tissues in and around the anal canal.

In a preferred embodiment, the vector construct is administered topically as an ointment, cream, lotion or solution.

In a second aspect, the present invention provides an isolated nucleotide sequence encoding at least one isoform of nitric oxide synthase (NOS) for use in the manufacture of a medicament for the treatment of an anal disease.

### PREFERRED MODE OF CARRYING OUT THE INVENTION

Nitric oxide (NO) is a small free radicle synthesised from the amino acid L-arginine by a family of enzymes, the nitric oxide synthases. NO is involved in many physiological and pathological processes and is produced in large amounts by an inducible isoform of NOS (iNOS) in cells of the immune system during host defence, immunological reactions and septic shock. Calcium dependent isoforms of the enzyme NOS are found in the brain(bNOS) and the endothelial layer (eNOS) of blood vessels. The calcium dependent isoforms of the enzyme are believed to release NO at low levels and maintain a relatively stable concentration of NO.

The following example outlines the general steps involved in the method of the present invention.

### EXAMPLE 1

1) Preparing a cDNA Clone of an Isoform of NOS Purification of NOS

A suitable cell culture is grown using the particular optimal conditions for that cell. For example, to purify inducible NOS, macrophage cells may be grown at 37° C., 5% CO₂ in 6 litres of RPMI 1640 (KC Biological Inc.) supplemented with 8% bovine calf serum (HyClone Systems), L-glutamine, penicillin and streptomycin.

When the cells reach the desired density, NOS activity is induced. Specifically, it is known that “mRNA is weakly induced following stimulation with cytokine signals such as tumour necrosis factor (TNF), interleukin-1 (IL-1) or interferon-gamma (IFN-g) (eg recombinant mouse IFN-g; Genentech). To further induce NOS activity, a bacterial lipopolysaccharide may be added (eg Escherichia lipopolysaccharide).

The cells are then harvested by centrifugation and resuspended in ice cold saline and glucose. The cells are repelleted and resuspended in cold water containing protease inhibitors and lysed by rapid freeze thawing. The lysate is separated by centrifugation and the supernatant fluid which contains NOS activity is stored.

The supernatant is chromatographed to eluted NOS activity and the active fragments concentrated, washed and stored at −80° C.

The sample is subjected to size exclusion gel filtration chromatography and the eluted purified enzyme stored.

2) Constructing a NOS cDNA Library

A suitable library is constructed using know techniques for example see Maniatis et al., J. Molecular Cloning: A Laboratory Manual (Cold Spring Harbour Laboratory, Cold Spring Harbour, N.Y., 1982). Using such a technique, the cDNA's are inserted into a λ phage vector.

3) Screening the cDNA Library for NOS cDNA Clones

To screen the library, 1×10⁶ phage are incubated with bacteria for a period of time so as to allow for phage lysis of the bacteria. The plaques are then transferred to nylon filters and positive clones identified by hybridization with a suitable cDNA probe.

The positive clones are rescued from the phage vector and transferred to another appropriate vector such as a plasmid vector. The latter vector enables the obtaining of a substantially full length cDNA clone encoding NOS.

4) Construction of a Recombinant Vector System and Introduction of the Vector into a Host Cell

The cDNA clone is ligated into a replication defective adenovirus vector and the recombinant vector together with a pharmaceutically acceptable carrier is transfected into the cells of the perianal region of laboratory rats by one of either injection or topical application of the vector to the skin of the perianal region.

The rats are sacrificed at one day, seven days and twenty one days following transfection and the NOS activity measured. The degree of expression of NOS in the cells of the rats when injected into the tissues and when topically applied to the skin is compared.

### Experiment 1

The experiment aimed to define the expression of NO synthase in the perianal region of the rat, the optimum method of achieving this and the time that the enzyme induction persists. The experiment also aimed to establish whether or not there was significant NO released locally and into the general circulation.

Methods

Laboratory rats were divided into three groups as follows:

Control group (n=4): paraffin paste applied to perianal region;

Trial group 1 (n=14): NO synthase inducer applied to the skin of the perianal region;

Trial group 2 (n=10): NO synthase inducer injected into the anal sphincter under general anaesthetic.

The rats of each group were euthanased at various time intervals as shown in Table 1.

Measurement was of the stable metabolic product of Nitric Oxide (NO) as NO itself is too unstable to measure readily in a large number of animals.

Results

The results shown in Table 1 indicate that application of the NOS induction code results in a significant increase in NO production locally. Further experimentation is required to determine the timeframe of action and compare the efficacy with commercially available products.

TABLE 1

| | | | pmol nitrate/ | |
|---|---|---|---|---|
| | pmol nitrate | mg tissue | 100 mg tissue | Time |
| Control | | | | |
| 3a | 35.09 | 36.35 | 96.53370014 | 1 day |
| 4a | 39.25 | 54.25 | 72.35023041 | 1 day |
| 4b | 35.56 | 34.75 | 102.3309353 | 1 day |
| 2a | 39.56 | 47.2 | 83.81355932 | 3 week |
| Topical Vector | | | | |
| 5a | 21.81 | 18.5 | 117.8918919 | 1 day |
| 5b | 157.15 | 64.55 | 243.4546863 | 1 day |
| 6a | 22.16 | 17.7 | 125.1977401 | 1 day |
| 6b | 26.77 | 31.7 | 84.44794953 | 1 day |
| 7a | 152.45 | 36.95 | 412.5845737 | 2 day |
| 7b | 78.05 | 19.65 | 397.2010178 | 2 day |
| 8b | 28.15 | 19.5 | 144.3589744 | 2 day |
| 9a | 34.82 | 30.4 | 114.5394737 | 1 week |
| 10a | 48.52 | 51.6 | 94.03100775 | 1 week |
| 10b | 42.56 | 29 | 146.7586207 | 1 week |
| 11a | 34.61 | 30.85 | 112.1880065 | 3 week |
| 11b | 51.34 | 47.85 | 107.2936259 | 3 week |
| 12a | 39.37 | 34.7 | 113.4582133 | 3 week |
| 12b | 28.40 | 34.9 | 81.37535817 | 3 week |
| Injected Vector | | | | |
| 13a | 53.88 | 56.15 | 95.95725735 | 1 day |
| 13b | 41.64 | 80.8 | 51.53465347 | 1 day |
| 14b | 50.71 | 74.05 | 68.48075625 | 1 day |
| 15a | 74.53 | 33.65 | 221.4858841 | 2 day |
| 16a | 65.59 | 31.7 | 206.9085174 | 2 day |
| 16b | 27.43 | 19.8 | 138.5353535 | 2 day |
| 17a | 79.46 | 32.95 | 241.1532625 | 1 week |
| 17b | 53.57 | 28.15 | 190.3019538 | 1 week |
| 18b | 43.14 | 38.9 | 110.8997429 | 1 week |
| 19b | 285.96 | 72.45 | 394.699793 | 3 week |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of treatment of an anal disease, said method including the step of increasing the level of at least one isoform of the enzyme nitric oxide synthase in the cells of the ano-rectal region of a subject in need of such treatment.

2. The method of claim 1 wherein upon increasing the level of nitric oxide synthase (NOS) activity in the cells of the ano-rectal region, cellular production of nitric oxide in the cells of said ano-rectal region is induced.

3. The method of claim 1 or claim 2 wherein the level of NOS in the cells of the ano-rectal region is increased by the introduction of a NOS enhancing factor to said cells.

4. The method of claim 3 wherein the NOS enhancing factor is a nucleotide sequence encoding at least one isoform of nitric oxide synthase.

5. The method of claim 4 wherein the nucleotide sequence is DNA.

6. The method of claim 4 wherein the nucleotide sequence is mRNA.

7. The method of any one of claims 4 to 6 wherein the nucleotide sequence encoding the at least one isoform of NOS is inserted into a vector.

8. The method of claim 7 wherein vector is suitable for introduction into the cells of the ano-rectal region of a subject.

9. The method of claim 8 wherein the vector is selected from the group comprising plasmids, avian retroviral vectors, murine retroviral vectors, adenoviral vectors, herpes viral vectors and non-replicative pox viruses.

10. The method of any one of claims 7 to 9 wherein the vector is replication defective.

11. The method of any one of claims 7 to 10 wherein the vector is an adenovirus.

12. The method of any one of claims 7 to 11 wherein the vector is introduced into the cells of the ano-rectal region by transformation or transfection.

13. The method of any one of claims 4 to 12 wherein the nucleotide sequence encoding the at least one isoform of NOS is introduced into the host cell by lipofection.

14. The method of any one of the preceding claims wherein the at least one isoform of NOS is an inducible isoform of NOS.

15. The method of any one of claims 1 to 13 wherein the at least one isoform of NOS is a constitutive isoform of NOS.

16. The method of claim 14 wherein the nucleotide sequence encodes vascular smooth muscle cell inducible NOS.

17. The method of claim 15 wherein the nucleotide sequence encodes brain cell constitutive NOS.

18. The method of claim 15 wherein the nucleotide sequence encodes vascular endothelial cell constitutive NOS.

19. The method of any one of the preceding claims wherein the level of the at least one isoform of NOS is increased by topically applying a formulation including the nucleotide sequence encoding the at least one isoform of nitric oxide synthase and a pharmaceutically acceptable carrier to the skin of the perianal region or the lining of the anal canal.

20. The method of claim 19 wherein the formulation is administered topically as an ointment, cream, lotion or solution.

21. The method of any one of claims 1 to 18 wherein the level of the at least one isoform of NOS is increased by injecting a formulation including the nucleotide sequence encoding the at least one isoform of nitric oxide synthase and a pharmaceutically acceptable carrier into the cells of the perianal region or the lining of the anal canal.

22. An isolated nucleotide sequence encoding at least one isoform of nitric oxide synthase (NOS) for use in the manufacture of a medicament for the treatment of an anal disease.
